(19) United States
(12) Patent Application Publication
(10) Pub. No.: US 2026/0256439 A1
(43) Pub. Date: Sep. 3, 2026

(54) RADIATION GENERATION APPARATUS

(71) Applicant: FUJIFILM CORPORATION, Tokyo (JP)

(72) Inventors: Hisatsugu HORIUCHI, Kanagawa (JP); Hiroaki KAWANO, Kanagawa (JP); Hidenori MATSUDA, Kanagawa (JP); Masayoshi MATSUURA, Kanagawa (JP)

(21) Appl. No.: 19/534,753

(22) Filed: Feb. 10, 2026

(30) Foreign Application Priority Data
Feb. 28, 2025 (JP) 2025-031676

(51) Int. Cl. A61B 6/00; A61B 6/40

(52) U.S. Cl. CPC A61B 6/4458; A61B 6/4021; A61B 6/4452; A61B 6/547

(57) ABSTRACT

A radiation generation apparatus has a configuration in which a body part having an arm that holds, at a distal end, a radiation source that emits radiation toward a patient is mounted on a carriage unit having wheels. The radiation generation apparatus includes a rotation mechanism that allows the body part to be rotate around a vertical axis with respect to the carriage unit.

### CROSS-REFERENCE TO RELATED APPLICATIONS

The present application claims priority under 35 U.S.C. §119 to Japanese Patent Application No. 2025-031676, filed on February 28, 2025. The above application is hereby expressly incorporated by reference, in its entirety, into the present application.

### BACKGROUND

### 1. Technical Field

The disclosed technology relates to a radiation generation apparatus.

### 2. Description of the Related Art

In a medical field, a driving type radiation generation apparatus in which a body part having a radiation source that emits radiation toward a subject is mounted on a carriage unit having wheels is used. For example, the driving type radiation generation apparatus is used for so-called ward round imaging in which a patient as a subject is imaged while moving around a ward.

For example, JP2017-119173A discloses a driving type radiation generation apparatus. In the driving type radiation generation apparatus described in JP2017-119173A, an arm that holds the radiation source at a distal end is attached to the body part. The arm is rotatable around a vertical axis with respect to the body part.

### SUMMARY

For example, in a case where the driving type radiation generation apparatus reaches a target position at a side of a decubitus imaging table (bed), the radiation source may be disposed to be inclined with respect to a radiographic image detection device such as an electronic cassette in a top view. In such a case, the arm needs to be rotated around the vertical axis with respect to the body part to eliminate the inclination. However, the radiation source has a relatively large weight of several kilograms, and in a case where the arm that holds the radiation source at the distal end is rotated, the body part may move due to a reaction force, or the arm may not stop at an intended position due to inertia.

One embodiment according to the disclosed technology provides a radiation generation apparatus that can contribute to high-accuracy alignment between a radiation source and a radiographic image detection device.

A radiation generation apparatus according to the present disclosure comprises: a body part having an arm that holds, at a distal end, a radiation source that emits radiation toward a subject, the body part being mounted on a carriage unit having wheels; and a rotation mechanism that allows the body part to rotate around a vertical axis with respect to the carriage unit.

It is preferable that the radiation generation apparatus comprises a first locking mechanism that is capable of locking the rotation of the body part by the rotation mechanism.

It is preferable that the locking by the first locking mechanism is released in a case where a set condition is satisfied.

It is preferable that the locking by the first locking mechanism is released in a case where a self-position is a target position.

It is preferable that the radiation generation apparatus comprises an environment information sensor, and a processor, in which the processor creates map data of a surrounding environment while estimating the self-position from output data of the environment information sensor.

It is preferable that the radiation generation apparatus is capable of autonomous driving based on the map data.

It is preferable that the radiation generation apparatus comprises a camera, in which the locking by the first locking mechanism is released in a case where it is detected that a radiographic image detection device or the subject is shown in an image of the camera.

It is preferable that the locking by the first locking mechanism is released in a case where an operation instruction for releasing the locking is input.

It is preferable that the carriage unit has a second locking mechanism that is capable of locking rotation of the wheels.

It is preferable that the locking by the second locking mechanism is performed in a case where the locking by the first locking mechanism is released.

It is preferable that the first locking mechanism is an electromagnetic brake.

It is preferable that the radiation generation apparatus comprises a processor, in which the processor controls the rotation of the body part by the rotation mechanism based on an inclination angle between the radiation source and a radiographic image detection device, an imaging table, or the subject.

It is preferable that the processor derives a difference between an azimuth angle of the radiation source detected by a geomagnetic sensor and an azimuth angle of the imaging table as the inclination angle.

It is preferable that a first magnet is provided in the body part, and alignment between the radiation source and the imaging table is performed by attraction of the first magnet to a second magnet provided on the imaging table.

It is preferable that the rotation mechanism rotates around the vertical axis passing through at least any of a center of the body part or a focal point of the radiation.

According to the disclosed technology, it is possible to provide a radiation generation apparatus that can contribute to high-accuracy alignment between a radiation source and a radiographic image detection device.

### BRIEF DESCRIPTION OF THE DRAWINGS

Exemplary embodiments according to the technique of the present disclosure will be described in detail based on the following figures, wherein:

FIG. 1 is a diagram showing a state of decubitus imaging using a radiography system;

FIG. 2 is a diagram showing a state of upright imaging using the radiography system;

FIG. 3 is a diagram showing a rotation mechanism and a first locking mechanism;

FIG. 4 is a block diagram showing an electric configuration of the radiation generation apparatus and a second locking mechanism;

FIG. 5 is a diagram showing a state in which the radiation generation apparatus drives from a standby position toward a first target position in the decubitus imaging and stops at the first target position;

FIG. 6 is a diagram showing a state in which the radiation generation apparatus drives from a standby position toward a second target position in the upright imaging and stops at the second target position;

FIG. 7 is a diagram showing processing of each processing unit of a processor related to driving control;

FIG. 8 is a diagram showing processing of each processing unit of the processor related to the driving control;

FIG. 9 is a diagram showing a case where the self-position is the target position, the driving control is transitioned to alignment control, the rotation of the body part by the first locking mechanism is unlocked, and the wheels are locked by the second locking mechanism;

FIG. 10 is a diagram showing processing of each processing unit of the processor related to the alignment control;

FIG. 11 is a diagram showing processing in a learning phase of a cassette contour extraction model;

FIG. 12 is a diagram showing a state of the alignment control;

FIG. 13 is a flowchart showing an imaging procedure;

FIG. 14 is a diagram showing a case where the electronic cassette is detected from an alignment control image, the driving control is transitioned to the alignment control, the rotation of the body part by the first locking mechanism is unlocked, and the wheels are locked by the second locking mechanism;

FIG. 15 is a diagram showing a case where the patient is detected from the alignment control image, the driving control is transitioned to the alignment control, the rotation of the body part by the first locking mechanism is unlocked, and the wheels are locked by the second locking mechanism;

FIG. 16 is a diagram showing a case where a rotation lock release button is operated, the rotation of the body part by the first locking mechanism is unlocked, and the wheels are locked by the second locking mechanism;

FIG. 17 is a diagram showing an aspect in which a joint point of the patient is extracted from the alignment control image;

FIG. 18 is a diagram showing an inclination angle in the aspect shown in FIG. 17;

FIG. 19 is a diagram showing an aspect in which an inclination angle is detected based on an azimuth angle detection result from a geomagnetic sensor;

FIG. 20 is a diagram showing an aspect in which the inclination angle is detected based on the azimuth angle detection result from the geomagnetic sensor;

FIG. 21 is a diagram showing an aspect in which the alignment between a radiation source and a decubitus imaging table is performed by a magnet;

FIG. 22 is a diagram showing an aspect in which the body part is rotated around a vertical axis passing through a focal point of radiation; and

FIG. 23 is a diagram showing an imaging table to which the rotation mechanism is applied.

### DETAILED DESCRIPTION

### First Embodiment

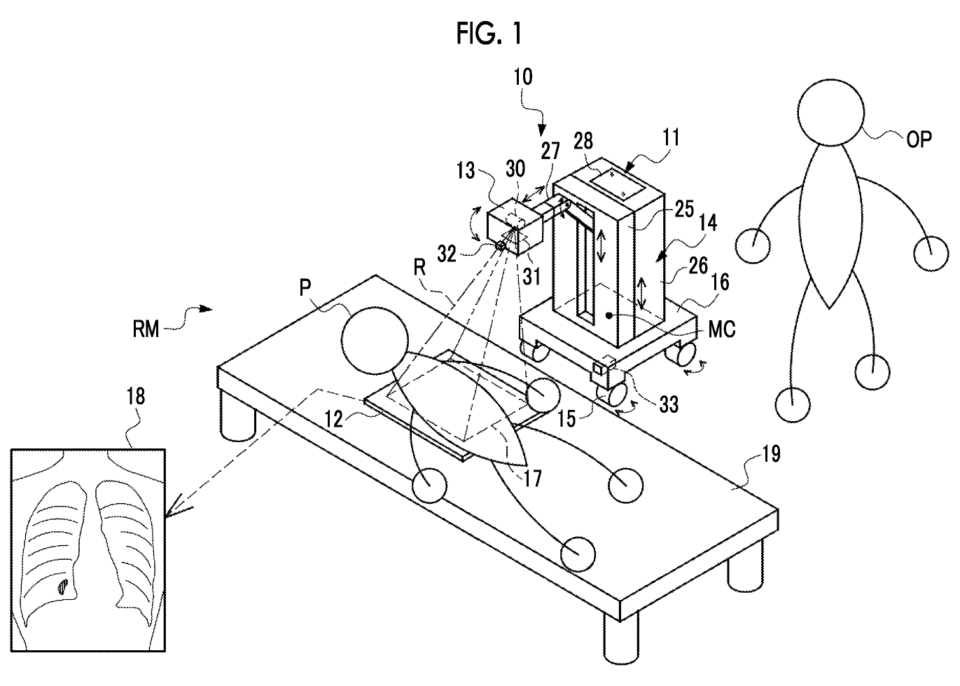

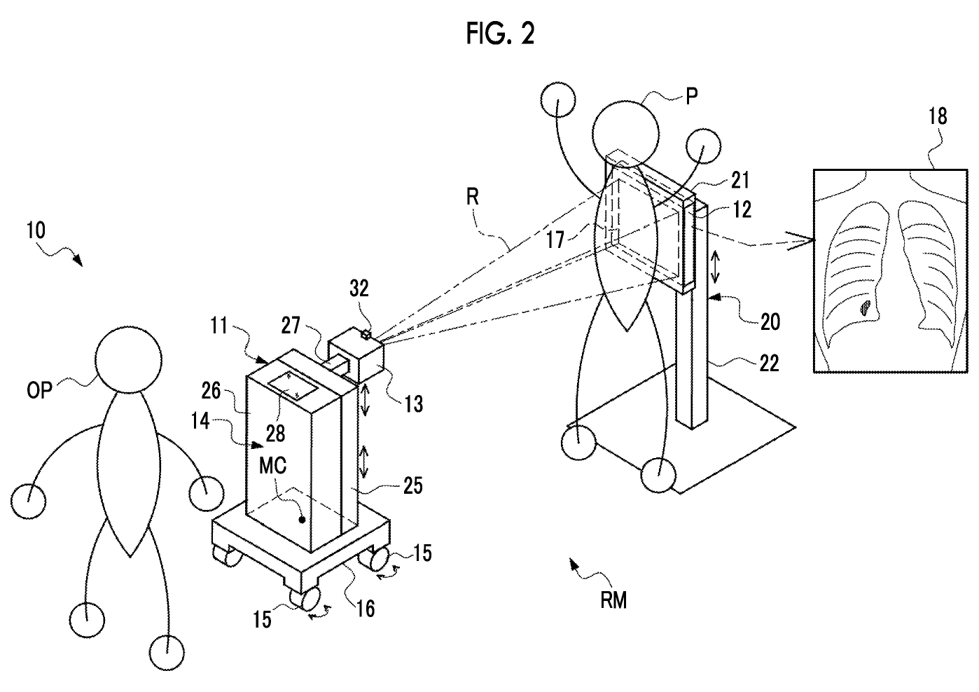

As shown in FIGS. 1 and 2 as an example, a radiography system 10 comprises a radiation generation apparatus 11 and an electronic cassette 12. The radiation generation apparatus 11 has a configuration in which a body part 14 having a radiation source 13 that emits radiation R toward a patient P is mounted on a carriage unit 16 having wheels 15. The radiation R is, for example, X-rays. A battery is mounted in the radiation generation apparatus 11, and the radiation generation apparatus 11 can be driven by being supplied with power from the battery. In addition, the radiation generation apparatus 11 can also be driven while being supplied with power from a commercial power supply via a power supply cord. Further, the electronic cassette 12 is an example of a “radiographic image detection device” according to the technology of the present disclosure. The patient P is an example of a "subject" according to the technology of the present disclosure.

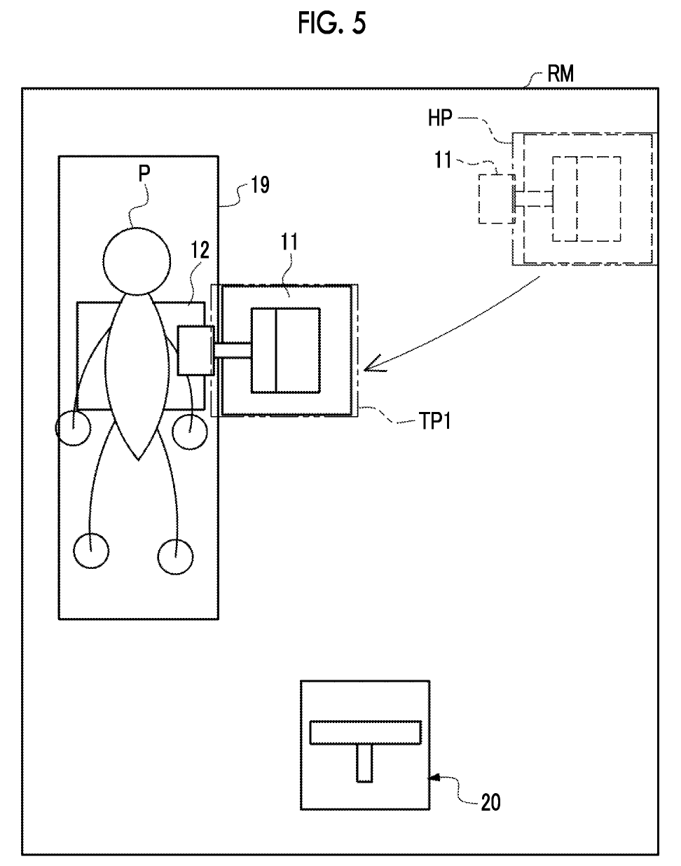

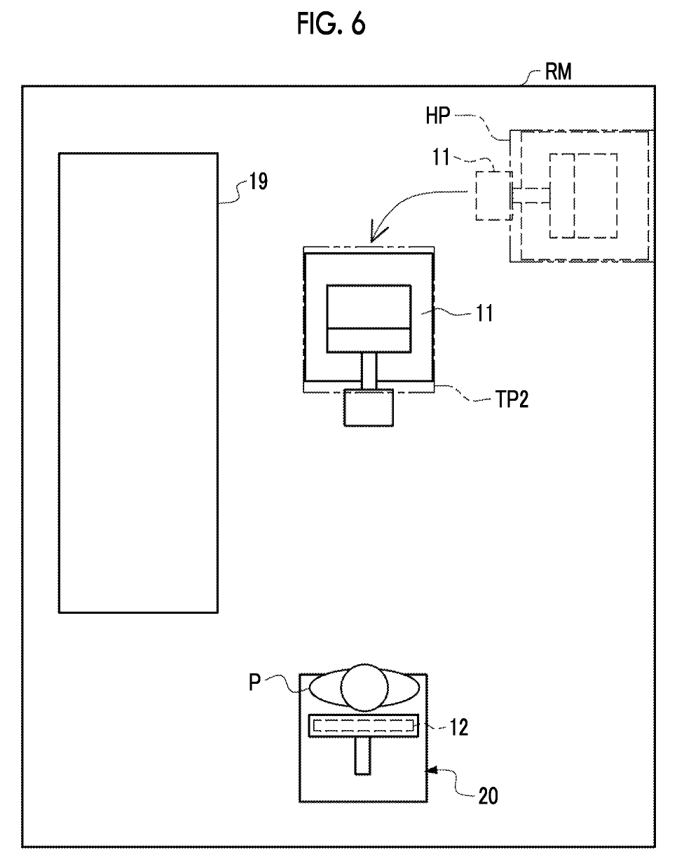

The radiation generation apparatus 11 is movable in an imaging room RM (see also FIGS. 5 and 6). In addition, the radiation generation apparatus 11 is used for so-called ward round imaging in which the patient P is imaged while moving around a ward. Therefore, the radiation generation apparatus 11 is also called a ward round cart. Alternatively, the radiation generation apparatus 11 is also used for imaging in an emergency room. In addition, the radiation generation apparatus 11 can also be carried into an operating room and used during surgery.

The electronic cassette 12 has a configuration in which a sensor panel as a radiation detector that detects the radiation R is built in a portable housing. The electronic cassette 12 is driven by the battery and performs wireless communication with the radiation generation apparatus 11. The sensor panel has a detection surface 17 in which a plurality of pixels that generate signal charges in response to the radiation R or visible light converted from the radiation R by a scintillator are arranged in a matrix. The electronic cassette 12 detects the radiation R emitted from the radiation source 13 and transmitted through the patient P, and outputs a radiographic image 18 of the patient P.

Since the electronic cassette 12 is portable and wireless, as shown in FIG. 1, the electronic cassette 12 can be used for so-called free imaging in which the electronic cassette 12 is placed on a decubitus imaging table (bed) 19 (under the patient P) installed in the imaging room RM to perform radiography. More specifically, FIG. 1 shows a state in which the electronic cassette 12 is inserted between the decubitus imaging table 19 and the patient P to image the patient P lying on the decubitus imaging table 19 (see also FIG. 5). In addition, as shown in FIG. 2, the electronic cassette 12 can also be used by being accommodated in a holder 21 of an upright imaging table 20 installed in the imaging room RM (see also FIG. 6). The holder 21 is movable up and down with respect to a support column 22.

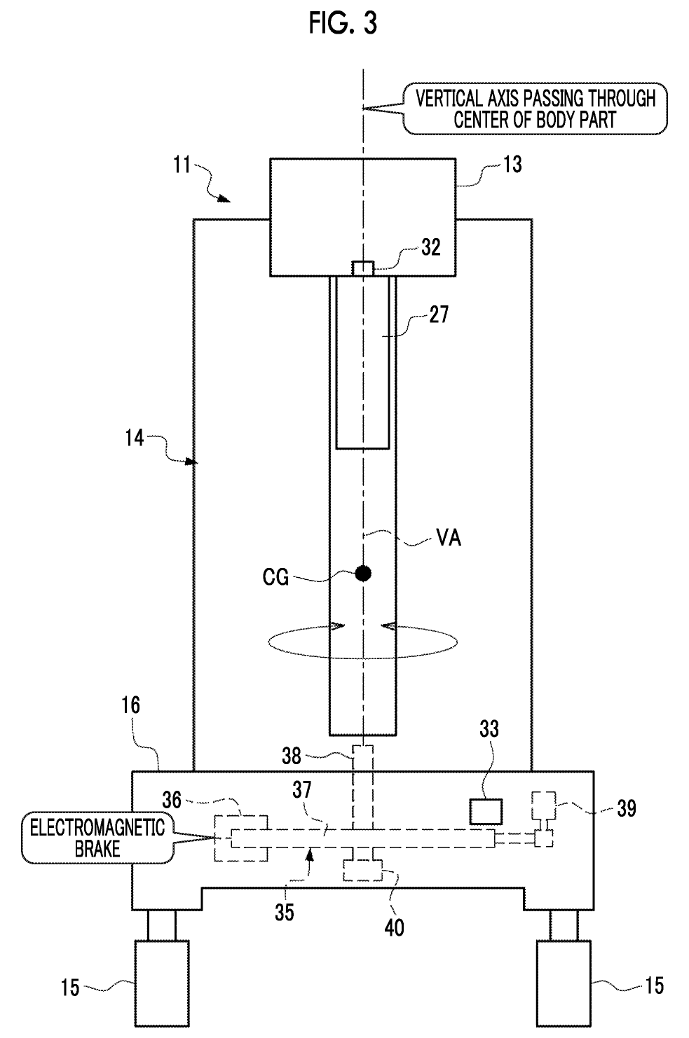

The body part 14 has a rectangular parallelepiped shape, and a center MC thereof matches a center of the carriage unit 16. That is, the body part 14 is erected at a center of the carriage unit 16. In addition, a centroid CG (see FIG. 3) of the radiation generation apparatus 11 is in the body part 14. The body part 14 is divided into a front part 25 and a rear part 26. The front part 25 is movable up and down with respect to the rear part 26.

A base end of an arm 27 is attached to the front part 25. More specifically, the arm 27 is divided into a first portion in which the base end is attached to the front part 25 and a second portion in which the base end is attached to the first portion. The radiation source 13 is attached to a distal end of the second portion that is a free end opposite to the base end.

The first portion is movable up and down with respect to the front part 25 and is bendable up and down with respect to the front part 25. The second portion is bendable up and down with respect to the first portion. Further, the second portion is expandable and contractible. The radiation source 13 is rotatable with respect to the second portion, that is, is swingable. By the displacement of the arm 27 and the displacement of the radiation source 13 with respect to the arm 27, a height position, a horizontal position, and a posture (orientation) of the radiation source 13 can be adjusted.

An operation panel 28 is provided on an upper surface of the rear part 26. The operation panel 28 is configured by, for example, a touch panel display and has a function of displaying information in addition to an operation function. The operation panel 28 is operated by an operator OP such as a medical radiologist. The operator OP sets an irradiation condition of the radiation R through the operation panel 28. In addition, the operator OP checks the radiographic image 18 through the operation panel 28.

In addition, an irradiation switch (not shown) is provided in the rear part 26. The irradiation switch is a switch that is provided to allow the operator OP to give an instruction to start irradiation of radiation. An extension cable is connected to the irradiation switch, and can be detached from the center portion 26 for use. The irradiation switch is operable only after driving control and alignment control, which will be described below, are completed.

The radiation source 13 includes a radiation tube 30 and an irradiation field limiter 31. The radiation tube 30 generates the radiation R. The radiation tube 230 is provided with a filament, a target, a grid electrode, and the like (all are not illustrated). A tube voltage is applied between the filament that is a cathode and the target that is an anode from a voltage generator (not shown) built in the rear part 26. The filament releases thermal electrons according to the applied tube voltage toward the target. The target radiates the radiation R with collision of the thermoelectrons released from the filament. The grid electrode is disposed between the filament and the target, and changes a flow rate of the thermoelectrons from the filament toward the target in response to the voltage applied from the voltage generator. The flow rate of the thermal electrons from the filament toward the target is referred to as a tube current. The tube voltage and the tube current are set to the radiation source 13 as the irradiation condition as the irradiation time elapses.

The irradiation field limiter 31 is also called a collimator and limits an irradiation field of the radiation R generated from the radiation tube 30. For example, the irradiation field limiter 31 has a configuration in which four shield plates formed of lead or the like shielding radiation R are disposed on respective sides of a quadrangle, and an emission opening of the quadrangle transmitting radiation is formed in a center portion. The irradiation field limiter 31 changes a size of the emission opening by changing a position of each shielding plate, thereby changing the irradiation field of the radiation R.

A camera 32 is attached to the radiation source 13. The camera 32 is used to support the alignment between the radiation source 13 and the electronic cassette 12. The camera 32 incorporates an imaging element that is sensitive to visible light, for example, a complementary metal oxide semiconductor (CMOS) image sensor or a charge-coupled device (CCD) image sensor. The camera 32 captures a video image at a predetermined frame rate. The camera 32 is an example of the "camera" according to the technology of the present disclosure.

The wheels 15 are provided four in total in front, rear, left, and right of the carriage unit 16. That is, the carriage unit 16 is a four-wheel type. Each wheel 15 is, for example, a revolution type that revolves around a revolution axis extending in a height direction (also referred to as a vertical direction) orthogonal to a rotation axis in a case of driving and rotating. The body part 14 autonomously drives by the carriage unit 16. The wheels 15 may be holonomic wheels such as omni-wheels.

Here, the autonomous driving refers to autonomously driving toward a set target position while estimating a self-position while recognizing a surrounding environment by using the environment information sensor. For example, a position at a side of the decubitus imaging table 19 shown in FIG. 1 and a position facing the upright imaging table 20 shown in FIG. 2 are set in advance as the target position. In the radiation generation apparatus 11, a simultaneous localization and mapping (SLAM) technology is used to realize the autonomous driving.

A camera 33 is provided in the carriage unit 16. The camera 33 is used to support the autonomous driving of the body part 14. The camera 33 images a front side of the carriage unit 16. The camera 33 also incorporates an imaging element that is sensitive to visible light and captures a video image at a predetermined frame rate, as in the camera 32. The camera 33 is an example of an "environment information sensor" according to the disclosed technology. The body part 14 and the radiation generation apparatus 11 can also be manually drived by the operator OP, in addition to the autonomous driving.

As shown in FIG. 3 as an example, a rotation mechanism 35 and a first locking mechanism 36 are built in the carriage unit 16. The rotation mechanism 35 and the first locking mechanism 36 are present below the centroid CG of the body part 14. The rotation mechanism 35 includes a rotation disk 37, a rotation shaft 38, a motor 39, and a rotary encoder 40. The rotation shaft 38 is inserted and fixed to a center of the rotation disk 37. In addition, the motor 39 is connected to the rotation disk 37 via a gear or the like. The rotation shaft 38 matches a vertical axis VA passing through the center MC of the body part 14. The rear part 26 of the body part 14 is attached to a distal end of the rotation shaft 38. Therefore, the motor 39 is driven to rotate the rotation disk 37 and the rotation shaft, so that the body part 14 rotates around the vertical axis VA with respect to the carriage unit 16. The rotary encoder 40 detects a rotation direction and a rotation amount of the rotation shaft 38 and the body part 14.

The first locking mechanism 36 is an electromagnetic brake that acts on the rotation disk 37. Specifically, the first locking mechanism 36 is a spring-type electromagnetic brake and includes an electromagnetic coil, an armature, a brake lining, a brake disk, a spring, and the like. In the first locking mechanism 36, a magnetic field is generated by energizing the electromagnetic coil, and the armature is separated from the brake disk. In this state, a frictional force is not generated between the brake lining and the rotation disk 37, and the rotation disk 37 can freely rotate. On the other hand, in the first locking mechanism 36, in a case where the energization of the electromagnetic coil is cut off, the magnetic field of the electromagnetic coil disappears, and the armature is pressed against the brake disk by a biasing force of the spring. As a result, a frictional force is generated between the brake lining and the rotation disk 37, and the rotation of the rotation disk 37 is locked. With the first locking mechanism 36, a state in which the rotation of the body part 14 is allowed and a state in which the rotation of the body part 14 is locked can be switched. The first locking mechanism 36 may be a pad brake. In addition, the first locking mechanism 36 may be configured by a hole formed in the rotation disk 37 and a pin that is inserted into and removed from the hole.

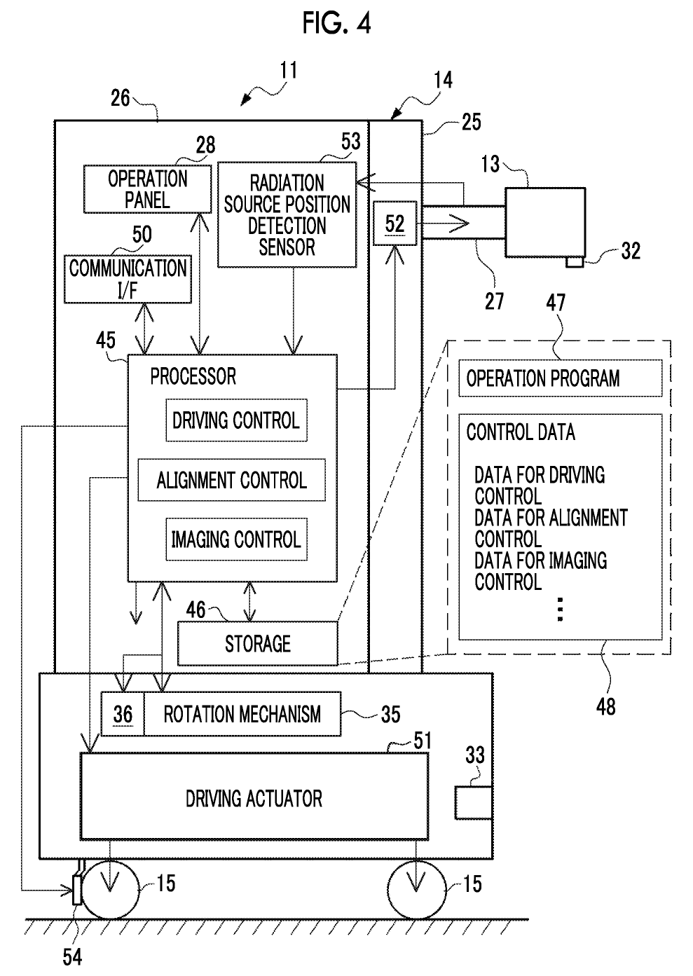

FIG. 4 is a block diagram showing an example of an electric configuration of the radiation generation apparatus 11. A processor 45 integrally controls the entire radiation generation apparatus 11. The processor 45 is configured by, for example, a central processing unit (CPU) and a memory such as a random access memory (RAM), and functions as various processing units by loading and executing various programs in the memory. Specifically, the processor 45 executes driving control, alignment control, and imaging control. The driving control is control related to the autonomous driving of the body part 14. The alignment control is control related to the alignment between the radiation source 13 and the electronic cassette 12. The imaging control includes irradiation control of the radiation R by the radiation source 13 and output control of the radiographic image 18 by the electronic cassette 12.

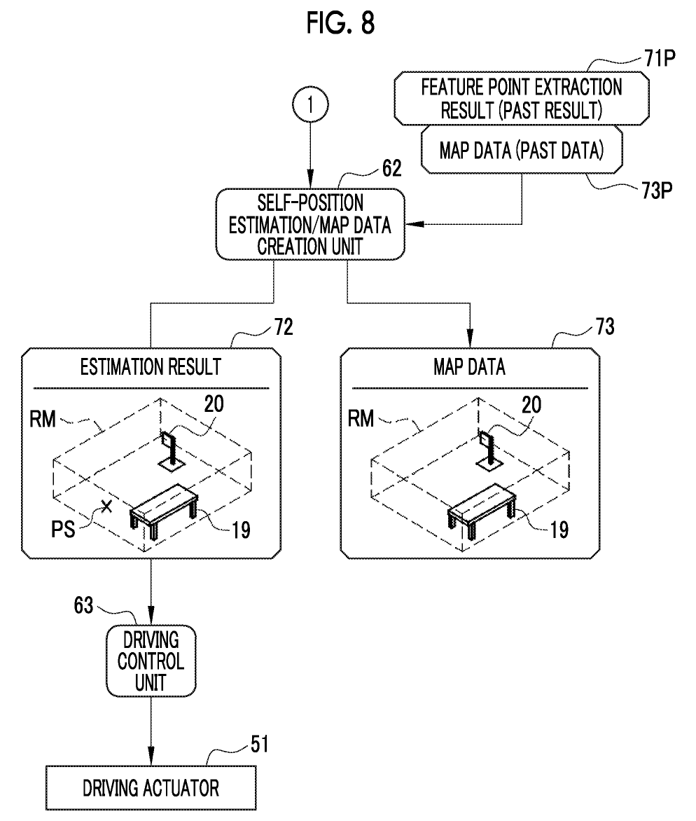

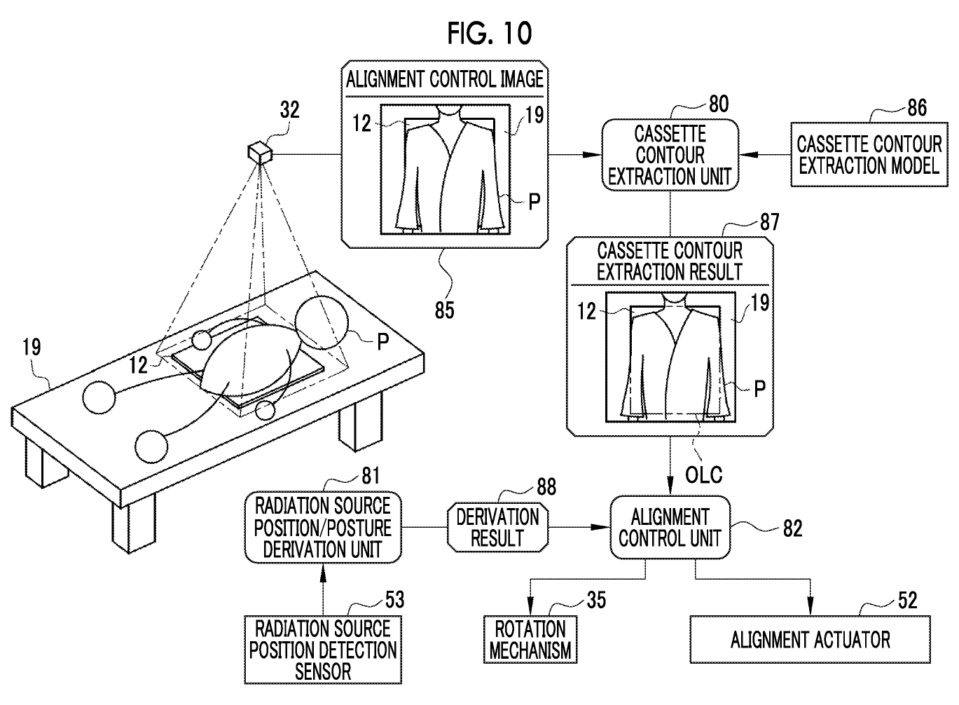

A storage 46 is configured by a non-volatile memory such as a hard disk drive or a solid state drive. The storage 46 stores an operation program 47 and control data 48. The control data 48 includes data for driving control, data for alignment control, and data for imaging control. Examples of the data for driving control include map data 73 (see FIG. 8) created by the SLAM technology. Examples of the data for alignment control include a cassette contour extraction model 86 (see FIG. 10) for extracting a contour OLC (see FIG. 10) of the electronic cassette 12. Examples of the data for imaging control include an irradiation condition table in which an irradiation condition for each imaging part is registered.

The operation panel 28, the rotation mechanism 35, the first locking mechanism 36, a communication interface (I/F) 50, a driving actuator 51, an alignment actuator 52, a radiation source position detection sensor 53, and a second locking mechanism 54 are connected to the processor 45. The processor 45 performs display control of various screens on the operation panel 28. In addition, the processor 45 receives various operation instructions of the operator OP through the operation panel 28 and executes various controls in response to the various operation instructions. The communication I/F 50 is, for example, a wireless communication I/F and performs wireless communication with the electronic cassette 12.

The processor 45 controls driving of the rotation mechanism 35 and the first locking mechanism 36. Specifically, the processor 45 controls a rotation direction and a rotation amount of the motor 39 of the rotation mechanism 35. The processor 45 receives a detection result of the rotation direction and the rotation amount of the body part 14 from the rotary encoder 40 of the rotation mechanism 35. The processor 45 derives a position of the body part 14 based on the detection result. In addition, the processor 45 controls energization and de-energization of the electromagnetic coil of the first locking mechanism 36.

The driving actuator 51 includes a motor for driving rotation of the wheels 15 and a motor for revolution, under the control of the processor 45. The alignment actuator 52 includes a motor for moving the front part 25 up and down, a motor for moving the arm 27 up and down, a motor for bending the second portion of the arm 27, a motor for expanding and contracting the second portion, and a motor for rotating the radiation source 13 with respect to the second portion, under the control of the processor 45.

The radiation source position detection sensor 53 measures an elevation direction and an elevation amount of the arm 27 with respect to the front part 25, a bending direction and a bending amount of the second portion of the arm 27 with respect to the first portion, an expansion and contraction direction and an expansion and contraction amount of the second portion, and a rotation direction and a rotation amount of the radiation source 13 with respect to the second portion. The radiation source position detection sensor 53 is, for example, a rotary encoder, a potentiometer, a gyro sensor, or a combination of a plurality of types of these sensors. The radiation source position detection sensor 53 outputs the measurement value to the processor 45. The processor 45 derives a position and a posture of the radiation source 13 based on the measurement value of the radiation source position detection sensor 53.

The second locking mechanism 54 is a pad brake that acts on the rear wheels on the left and right of the four wheels 15. In the second locking mechanism 54, the actuator is driven to press the pad against an outer peripheral surface of the wheels 15 to generate a braking force. The processor 45 executes the locking and unlocking of the rotation of the wheels 15 by the second locking mechanism 54 by controlling the driving of the actuator. The second locking mechanism 54 is not limited to the pad brake and may be an electromagnetic brake such as the first locking mechanism 36.

As shown in FIGS. 5 and 6 as an example, a standby position HP of the radiation generation apparatus 11 is prepared in a corner of the imaging room RM. In the standby position HP, charging of the battery, receiving of an imaging order from a radiology information system (RIS), setting of the irradiation condition, and the like are performed. The standby position HP occupies a region equal to, or one size larger than, that of the radiation generation apparatus 11.

In the imaging room RM, a first target position TP1 (see FIG. 5) of the radiation generation apparatus 11 in the decubitus imaging and a second target position TP2 (see FIG. 6) of the radiation generation apparatus 11 in the upright imaging are set. The first target position TP1 is a position at a side of the decubitus imaging table 19, and more specifically, a position facing one long side of the decubitus imaging table 19. The second target position TP2 is a position facing the upright imaging table 20 and spaced from the upright imaging table 20 by a distance of an SID required for the upright imaging. The first target position TP1 and the second target position TP2 occupy a region equal to, or one size larger than, that of the radiation generation apparatus 11, like the standby position HP. In the following, the first target position TP1 and the second target position TP2 may be collectively referred to as a target position TP.

In a case of the decubitus imaging, the radiation generation apparatus 11 drives from the standby position HP toward the first target position TP1 and stops in a case where a self-position PS (see FIG. 8) reaches the first target position TP1 (see FIG. 5). In addition, in a case of the upright imaging, the radiation generation apparatus 11 drives from the standby position HP toward the second target position TP2 and stops in a case where the self-position PS reaches the second target position TP2 (see FIG. 6). Although not shown, the radiation generation apparatus 11 may drive from the first target position TP1 toward the second target position TP2 in order to perform the upright imaging after the decubitus imaging. In addition, the radiation generation apparatus 11 may drive from the second target position TP2 toward the first target position TP1 in order to perform the decubitus imaging after the upright imaging. In a case where the self-position PS matches the center of the target position TP, it may be determined that the self-position PS has reached the target position TP, or in a case where the self-position PS is within a range including the center of the target position TP with a certain margin, it may be determined that the self-position PS has reached the target position TP.

The autonomous driving to the target position TP is started, for example, by an instruction of the operator OP through the operation panel 28. In addition to or instead of the operation panel 28, a configuration may be adopted in which an instruction to start the autonomous driving can be issued by a remote controller.

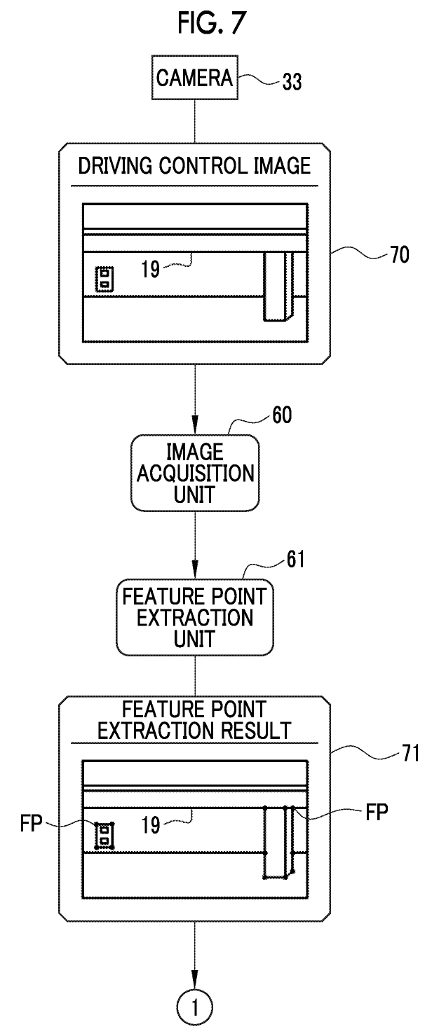

The processor 45 executes the driving control using the SLAM technology. Specifically, as shown in FIGS. 7 and 8, the processor 45 functions as an image acquisition unit 60, a feature point extraction unit 61, a self-position estimation/map data creation unit 62, and a driving control unit 63.

The image acquisition unit 60 sequentially acquires an image 70 (hereinafter, referred to as a driving control image) of the surrounding environment of the radiation generation apparatus 11 captured by the camera 33 at a predetermined frame rate. The image acquisition unit 60 performs preprocessing such as noise removal and distortion correction on the driving control image 70 and then outputs the driving control image 70 to the feature point extraction unit 61. The driving control image 70 is an example of "output data" according to the disclosed technology.

The feature point extraction unit 61 extracts a corner of a structure present in the surrounding environment shown in the driving control image 70 as a feature point FP by using an algorithm such as oriented features from accelerated segment test and rotated binary robust independent elementary features (ORB) or speeded-up robust features (SURF). The feature point extraction unit 61 outputs a feature point extraction result 71 to the self-position estimation/map data creation unit 62. In addition, although not shown, the feature point extraction unit 61 stores the feature point extraction result 71 in the storage 46. The feature point extraction result 71 is a set of coordinates of each feature point FP and a feature amount vector.

The self-position estimation/map data creation unit 62 estimates the self-position PS of the radiation generation apparatus 11 and creates map data 73 of the surrounding environment. The feature point extraction result 71 is input to the self-position estimation/map data creation unit 62 from the feature point extraction unit 61. In addition, a feature point extraction result (hereinafter, referred to as a feature point extraction result (past result)) 71P for a plurality of past frames and map data (hereinafter, referred to as map data (past data)) 73P for a plurality of past frames are input to the self-position estimation/map data creation unit 62.

The feature point extraction result (past result) 71P and the map data (past data) 73P are stored in the storage 46 as the data for driving control of the control data 48. The feature point extraction result 71 and the map data 73 constituting the feature point extraction result (past result) 71P and the map data (past data) 73P are, for example, for several tens to several hundreds of frames. The feature point extraction result (past result) 71P and the map data (past data) 73P include the feature point extraction result 71 and the map data 73 that are considered to play an important role in the estimation of the self-position PS and the creation of the map data 73. The feature point extraction result 71 and the map data 73 are, for example, the feature point extraction result 71 and the map data 73 obtained for each movement of a certain distance. In addition, for example, the feature point extraction result 71 and the map data 73 obtained in a case where a large viewpoint change has occurred from the previous frame. Further, for example, the feature point extraction result 71 and the map data 73 obtained in a case where a set amount or more of new feature points FP are extracted.

The self-position estimation/map data creation unit 62 matches the feature point FP of the feature point extraction result 71 from the feature point extraction unit 61 with the feature point FP of the feature point extraction result (past result) 71P. In this case, the self-position estimation/map data creation unit 62 refers to the feature amount vector of each feature point FP. More specifically, the feature point FP having a distance (Euclidean distance or the like) of the feature amount vector less than a threshold value between the feature point FP of the feature point extraction result 71 from the feature point extraction unit 61 and the feature point FP of the feature point extraction result (past result) 71P is recognized as the same feature point FP. The self-position estimation/map data creation unit 62 estimates the self-position PS based on the matching result of the feature point FP and the map data (past data) 73P. In addition, the self-position estimation/map data creation unit 62 creates (updates the map data 73) new map data 73 based on the feature point extraction result 71 from the feature point extraction unit 61, the feature point extraction result (past result) 71P, the map data (past data) 73P, and an estimation result 72. As described above, the self-position estimation/map data creation unit 62 estimates the self-position PS and creates the map data 73 in parallel in a process in which the radiation generation apparatus 11 autonomously drives. The self-position estimation/map data creation unit 62 does not estimate the self-position PS until a sufficient amount of the feature point extraction result (past result) 71P is accumulated, and only creates (updates) the map data 73.

The self-position estimation/map data creation unit 62 outputs the estimation result 72 of the self-position PS to the driving control unit 63. The driving control unit 63 controls the driving of the driving actuator 51 such that the self-position PS is the target position TP. In addition, although not shown, the self-position estimation/map data creation unit 62 stores the map data 73 in the storage 46. Specifically, the map data 73 is three-dimensional data of the imaging room RM including a structure such as the decubitus imaging table 19 and the upright imaging table 20. In addition, the standby position HP and the target position TP are registered in the map data 73.

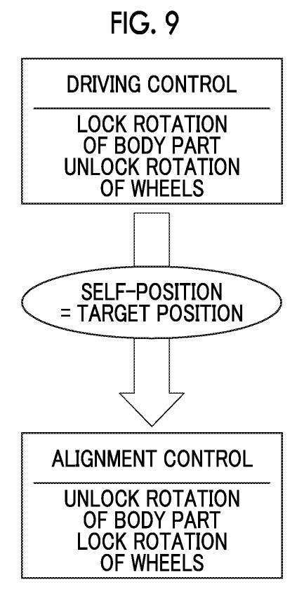

In a case where the driving actuator 51 is driven under the control of the driving control unit 63 and the self-position PS of the radiation generation apparatus 11 is the target position TP, as shown in FIG. 9 as an example, the processor 45 transitions from the driving control to the alignment control. While the driving control is being executed, the processor 45 locks the rotation of the body part 14 by the first locking mechanism 36 and unlocks the locking of the rotation of the wheels 15 by the second locking mechanism 54. On the other hand, in a case where the self-position PS of the radiation generation apparatus 11 is the target position TP and the driving control is transitioned to the alignment control, the processor 45 unlocks the rotation of the body part 14 by the first locking mechanism 36 and locks the rotation of the wheels 15 by the second locking mechanism 54. In a case where the self-position PS of the radiation generation apparatus 11 is the target position TP, it is an example of a "case where a set condition is satisfied" according to the disclosed technology.

As shown in FIG. 10 as an example, the processor 45 functions as a cassette contour extraction unit 80, a radiation source position/posture derivation unit 81, and an alignment control unit 82.

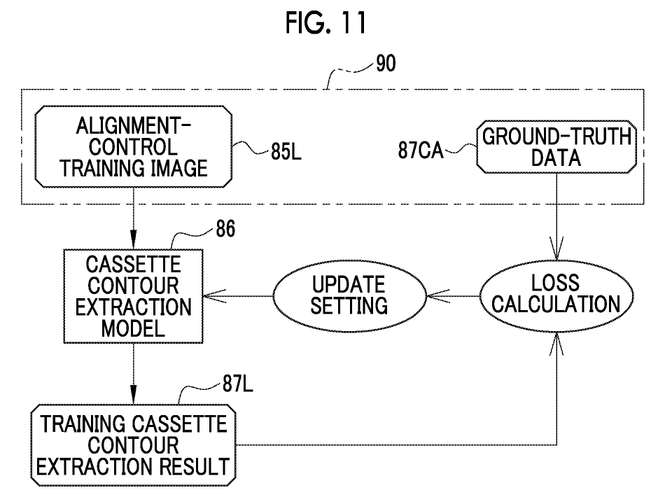

An image 85 (hereinafter, referred to as an alignment control image) including the patient P and the electronic cassette 12 captured by the camera 32 at a predetermined frame rate is sequentially input to the cassette contour extraction unit 80. In a case where the self-position PS of the radiation generation apparatus 11 is the target position TP, the patient P and the electronic cassette 12 are shown in the alignment control image 85. In FIG. 11, since the decubitus imaging is illustrated, the decubitus imaging table 19 is also shown in the alignment control image 85. The alignment control image 85 is an example of a "image of the camera" according to the disclosed technology. It is assumed that the alignment between the patient P and the electronic cassette 12 is performed by the operator OP before the driving control and the alignment control.

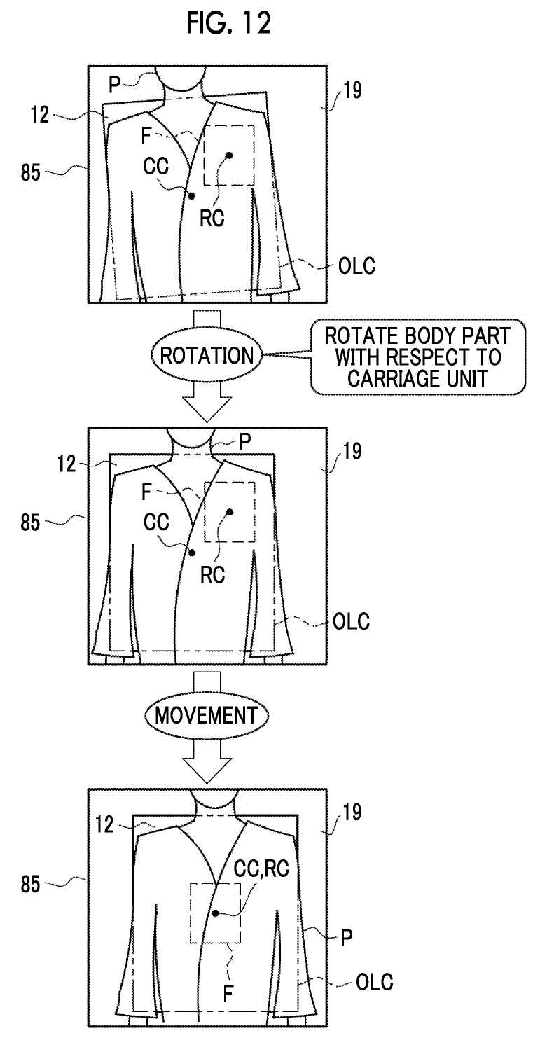

The cassette contour extraction unit 80 extracts the contour OLC of the electronic cassette 12 from the alignment control image 85 by using the cassette contour extraction model 86. According to the contour OLC, a center CC (see FIG. 12) of the detection surface 17 of the electronic cassette 12 and a posture of the electronic cassette 12 with respect to the radiation source 13 are known. The cassette contour extraction unit 80 outputs a cassette contour extraction result 87 to the alignment control unit 82. Here, a contour following all sides of the electronic cassette 12 is illustrated as the contour OLC, but the present disclosure is not limited thereto. Four corners of the electronic cassette 12 may be extracted as the contour.

The cassette contour extraction model 86 is a trained model that is configured by, for example, a convolutional neural network and is trained to output the cassette contour extraction result 87 in a case where the alignment control image 85 is input. The cassette contour extraction model 86 is stored in the storage 46 as the data for alignment control of the control data 48.

As shown in FIG. 11 as an example, in the learning phase, learning data 90 is given to the cassette contour extraction model 86. The learning data 90 is composed of a set of a alignment-control training image 85L and ground-truth data 87CA. The electronic cassette 12 is shown in the alignment-control training image 85L. The ground-truth data 87CA is data in which the contour OLC of the electronic cassette 12 shown in the alignment-control training image 85L is annotated, and is data for checking the answer.

The alignment-control training image 85L is input to the cassette contour extraction model 86. The cassette contour extraction model 86 outputs a training cassette contour extraction result 87L in response to the input of the alignment-control training image 85L. A loss calculation of the cassette contour extraction model 86 using a loss function is performed based on the training cassette contour extraction result 87L and the ground-truth data 87CA. Then, update settings of various coefficients (coefficients of filters of a convolutional layer and the like) of the cassette contour extraction model 86 are made according to the result of the loss calculation, and the cassette contour extraction model 86 is updated according to the update settings.

In the learning phase of the cassette contour extraction model 86, the series of processing of the input of the alignment-control training image 85L to the cassette contour extraction model 86, the output of the training cassette contour extraction result 87L from the cassette contour extraction model 86, the loss calculation, the update setting, and the update of the cassette contour extraction model 86 is repeatedly performed while the learning data 90 is replaced. The repetition of the series of processing is ended in a case where the extraction accuracy of the training cassette contour extraction result 87L reaches a predetermined set level. The cassette contour extraction model 86 in which the extraction accuracy reaches the set level is stored in the storage 46 and is used by the cassette contour extraction unit 80. The learning may be ended in a case where the series of processing is repeated a set number of times regardless of the extraction accuracy of the training cassette contour extraction result 87L.

The radiation source position/posture derivation unit 81 derives the position and the posture of the radiation source 13 based on the measurement value of the radiation source position detection sensor 53. The radiation source position/posture derivation unit 81 outputs a derivation result 88 to the alignment control unit 82. The derivation result 88 is coordinates of an irradiation center RC (see FIG. 12) of the radiation R in the alignment control image 85 and coordinates of a rectangular frame F (see FIG. 12) indicating the posture of the radiation source 13 in the alignment control image 85. Since the positional relationship between the radiation source 13 and the camera 32 is known, the coordinates of the irradiation center RC and the frame F in the alignment control image 85 can be easily calculated from the measurement value of the radiation source position detection sensor 53.

The alignment control unit 82 controls the driving of the rotation mechanism 35 and the alignment actuator 52 such that the radiation source 13 and the electronic cassette 12 face each other. More specifically, as shown in FIG. 12 as an example, in a case where the posture of the radiation source 13 is inclined with respect to the electronic cassette 12, the alignment control unit 82 drives the rotation mechanism 35 to rotate the body part 14, thereby rotating the radiation source 13 to eliminate the inclination. Specifically, in a case where the inclination angle of the radiation source 13 with respect to the electronic cassette 12 is θ, the alignment control unit 82 rotates the radiation source 13 such that the inclination angle θ is 0. The inclination angle θ is an angle between the radiation source 13 and the electronic cassette 12 around a normal line of the detection surface 17. Furthermore, the inclination angle θ is an angle between a side extending to the left and right of the radiation source 13 and a long side of the detection surface 17 around the normal line of the detection surface 17.

In addition, in a case where the irradiation center RC of the radiation R and the center CC of the detection surface 17 of the electronic cassette 12 are shifted, the alignment control unit 82 moves the radiation source 13 to eliminate the shift. After the shift between the irradiation center RC of the radiation R and the center CC of the detection surface 17 of the electronic cassette 12 is eliminated, the inclination may be eliminated. In addition, in FIG. 12, a case where the posture of the radiation source 13 is inclined around the normal line of the detection surface 17 is illustrated, but the present disclosure is not limited thereto. Even in a case where the posture of the radiation source 13 is inclined around an axis along the long side of the detection surface 17 or around an axis along the short side of the detection surface 17, the alignment control unit 82 rotates the radiation source 13 to eliminate the inclination.

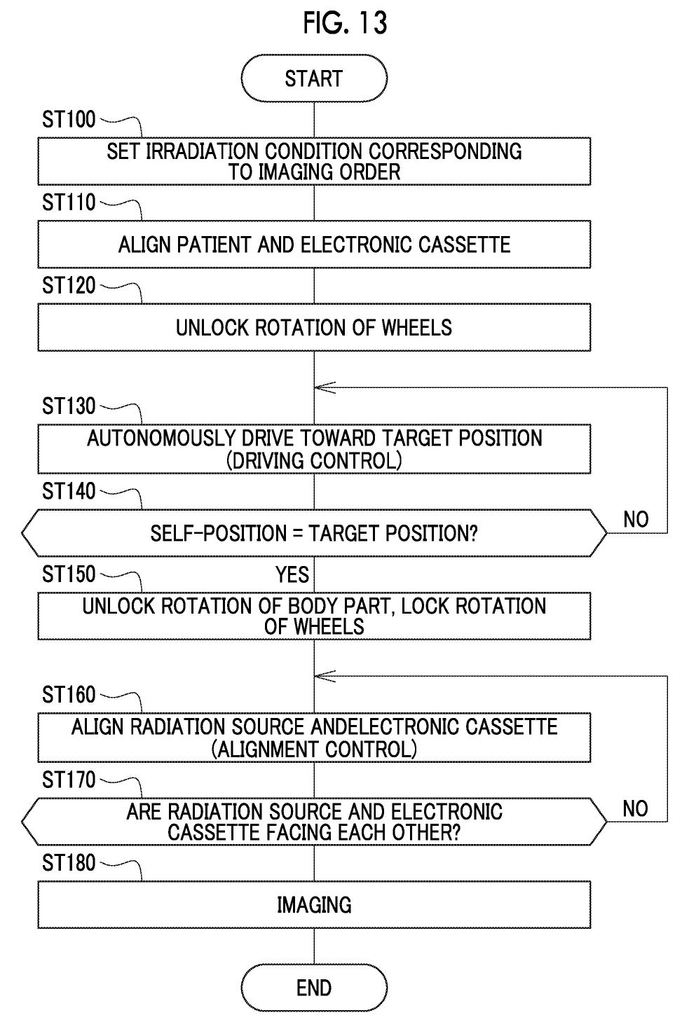

Next, an action with the configuration described above will be described with reference to the flowchart shown in FIG. 13 as an example. Before the imaging, the radiation generation apparatus 11 waits at the standby position HP in the imaging room RM. In this case, under the control of the processor 45, the rotation of the body part 14 is locked by the first locking mechanism 36, and the rotation of the wheels 15 is locked by the second locking mechanism 54.

An imaging order is transmitted from the radiology information system to the radiation generation apparatus 11. The operator OP operates the operation panel 28 to set the irradiation condition corresponding to the imaging order (step ST100).

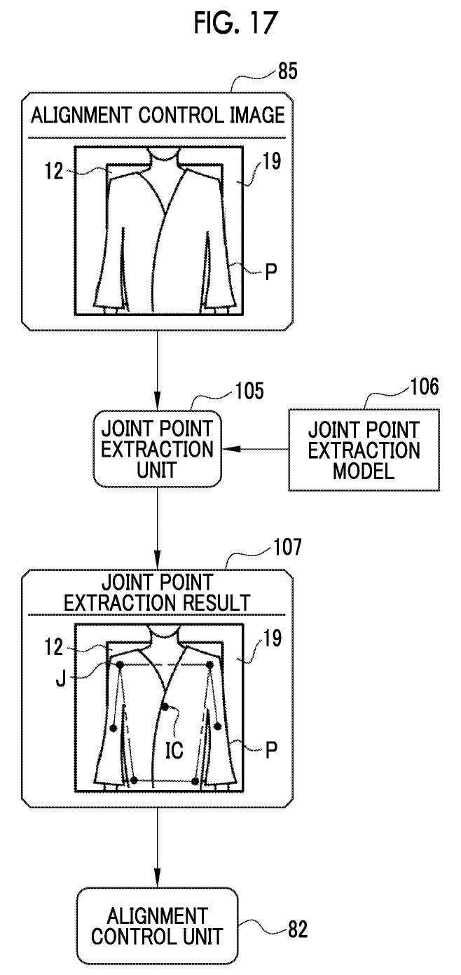

The operator OP places the electronic cassette 12 on the decubitus imaging table 19 in a case of the decubitus imaging, and accommodates the electronic cassette 12 in the holder 21 of the upright imaging table 20 in a case of the upright imaging. Then, the alignment between the patient P and the electronic cassette 12 is performed (step ST110). Specifically, the center IC (see FIG. 17) of the imaging part of the patient P and the center CC of the detection surface 17 of the electronic cassette 12 are aligned. In addition, the body axis (head-tail axis) of the patient P and the long side of the detection surface 17 of the electronic cassette 12 are made parallel to each other.

After the rotation of the wheels 15 by the second locking mechanism 54 is unlocked under the control of the processor 45 in response to the instruction of the operator OP (step ST120), the autonomous driving of the radiation generation apparatus 11 from the standby position HP toward the target position TP is started (step ST130). In the autonomous driving, the processor 45 executes the driving control using the SLAM technology, which is shown in FIGS. 7 and 8 as an example.

Specifically, first, the driving control image 70 captured by the camera 33 is acquired by the image acquisition unit 60. The driving control image 70 is output from the image acquisition unit 60 to the feature point extraction unit 61. Then, in the feature point extraction unit 61, the feature point FP of the structure present in the surrounding environment shown in the driving control image 70 is extracted. The feature point extraction result 71 is output from the feature point extraction unit 61 to the self-position estimation/map data creation unit 62. In addition, the feature point extraction result 71 is stored in the storage 46.

In the self-position estimation/map data creation unit 62, the self-position PS of the radiation generation apparatus 11 is estimated based on the feature point extraction result 71, the feature point extraction result (past result) 71P, and the map data (past data) 73P, and the estimation result 72 is output. In addition, in the self-position estimation/map data creation unit 62, the map data 73 is created based on the feature point extraction result 71, the feature point extraction result (past result) 71P, the map data (past data) 73P, and the estimation result 72. The estimation result 72 of the self-position PS is output from the self-position estimation/map data creation unit 62 to the driving control unit 63. In addition, the map data 73 is stored in the storage 46.

Under the control of the driving control unit 63, the driving of the driving actuator 51 is controlled such that the self-position PS is the target position TP. The driving control is executed in this way. The driving control is continued until the self-position PS of the radiation generation apparatus 11 is the target position TP (NO in step ST140).

In a case where the self-position PS of the radiation generation apparatus 11 is the target position TP (YES in step ST140), as shown in FIG. 9, under the control of the processor 45, the rotation of the body part 14 by the first locking mechanism 36 is unlocked, and the rotation of the wheels 15 is locked by the second locking mechanism 54 (step ST150). Then, the driving control is transitioned to the alignment control (step ST160).

As shown in FIG. 10, the alignment control image 85 captured by the camera 32 is input to the cassette contour extraction unit 80. In the cassette contour extraction unit 80, the contour OLC of the electronic cassette 12 is extracted from the alignment control image 85 by using the cassette contour extraction model 86. The cassette contour extraction result 87 is output from the cassette contour extraction unit 80 to the alignment control unit 82.

The radiation source position/posture derivation unit 81 derives the position and the posture of the radiation source 13 based on the measurement value of the radiation source position detection sensor 53. The derivation result 88 of the position and the posture of the radiation source 13 is output from the radiation source position/posture derivation unit 81 to the alignment control unit 82.

Under the control of the alignment control unit 82, the driving of the rotation mechanism 35 and the alignment actuator 52 is controlled such that the radiation source 13 and the electronic cassette 12 face each other. The alignment control is executed in this way. The alignment control is continued until the radiation source 13 and the electronic cassette 12 face each other (NO in step ST170).

In a case where the radiation source 13 and the electronic cassette 12 face each other (YES in step ST170), the irradiation switch can be operated. The operator OP operates the irradiation switch to issue an instruction to start the irradiation of the radiation. As a result, the radiation R is emitted from the radiation source 13, and the radiographic image 18 is output by the electronic cassette 12 (step ST180).

As described above, the radiation generation apparatus 11 has a configuration in which the body part 14 having the arm 27 that holds, at the distal end, the radiation source 13 that emits the radiation R toward the patient P is mounted on the carriage unit 16 having the wheels 15. The radiation generation apparatus 11 comprises the rotation mechanism 35 that allows the body part 14 to rotate around the vertical axis VA with respect to the carriage unit 16.

The radiation source 13 is located above the centroid CG of the body part 14. The radiation source 13 protrudes toward the patient P. Therefore, in a case where the arm 27 is rotated as in the related art, the body part 14 may move due to a reaction force, or the arm 27 may not stop at an intended position due to inertia. However, in the disclosed technology, the body part 14 is rotatable around the vertical axis VA with respect to the carriage unit 16 by the rotation mechanism 35. Therefore, the body part 14 does not move due to a reaction force, and the arm 27 does not stop at an unintended position due to inertia, so that the rotation of the radiation source 13 can be stabilized. Therefore, it is possible to contribute to the high-accuracy alignment between the radiation source 13 and the electronic cassette 12.

As shown in FIG. 3, the radiation generation apparatus 11 comprises the first locking mechanism 36 that can lock the rotation of the body part 14. Therefore, the unintended rotation of the body part 14 can be prevented, and the body part 14 can be rotated only when necessary. The body part 14 does not unintentionally rotate, and a situation in which the radiation source 13 collides with the operator OP can be avoided, so that the safety can be improved.

As shown in FIG. 9, in a case where the self-position PS is the target position TP, the locking by the first locking mechanism 36 is released. Therefore, the body part 14 can be rotated at an appropriate timing. The alignment control can be executed without a hitch.

As shown in FIGS. 7 and 8, in the driving control, the map data 73 of the surrounding environment is created while estimating the self-position PS from the driving control image 70 captured by the camera 33. Therefore, the self-position PS can be accurately estimated. The autonomous driving can be performed even in an unknown environment. In addition, the target position TP can be reached on an appropriate driving path such as a shortest path or a path avoiding an obstacle.

The driving control unit 63 executes the autonomous driving based on the map data 73. Therefore, the advanced autonomous driving can be realized, and the operator OP can be saved from the effort.

As shown in FIG. 4, the carriage unit 16 has the second locking mechanism 54 that can lock the rotation of the wheels 15. Therefore, the unintended rotation of the wheels 15 can be prevented, and the wheels 15 can be rotated only when necessary.

As shown in FIG. 9, in a case where the locking by the first locking mechanism 36 is released, the locking by the second locking mechanism 54 is performed. Therefore, it is possible to reduce the possibility that the carriage unit 16 moves due to the reaction force of the rotation of the body part 14 by the rotation mechanism 35. The rotation of the radiation source 13 can be further stabilized.

As shown in FIG. 3, the first locking mechanism 36 is an electromagnetic brake. Therefore, the rotation of the body part 14 can be locked with a relatively inexpensive and simple configuration.

As shown in FIG. 12, the alignment control unit 82 controls the rotation of the body part 14 by the rotation mechanism 35 based on the inclination angle θ between the radiation source 13 and the electronic cassette 12. Therefore, the operator OP can be saved from the effort.

As shown in FIG. 3, the rotation mechanism 35 rotates around the vertical axis VA passing through the center MC of the body part 14. Therefore, the rotation of the body part 14 and the radiation source 13 can be further stabilized.

### Second Embodiment

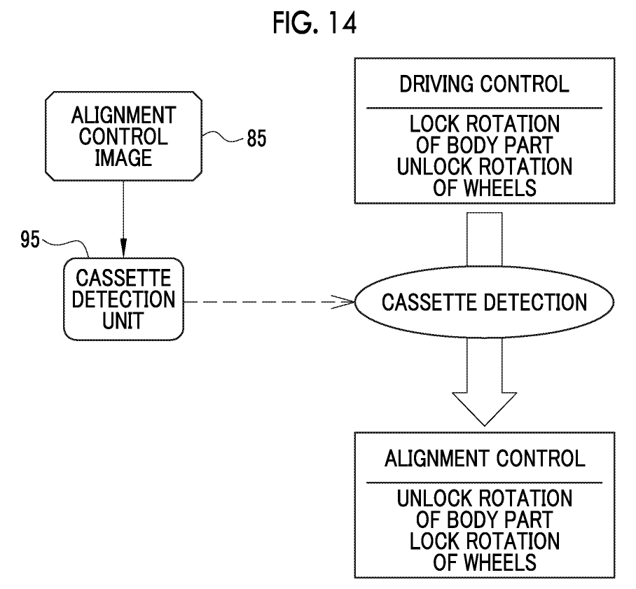

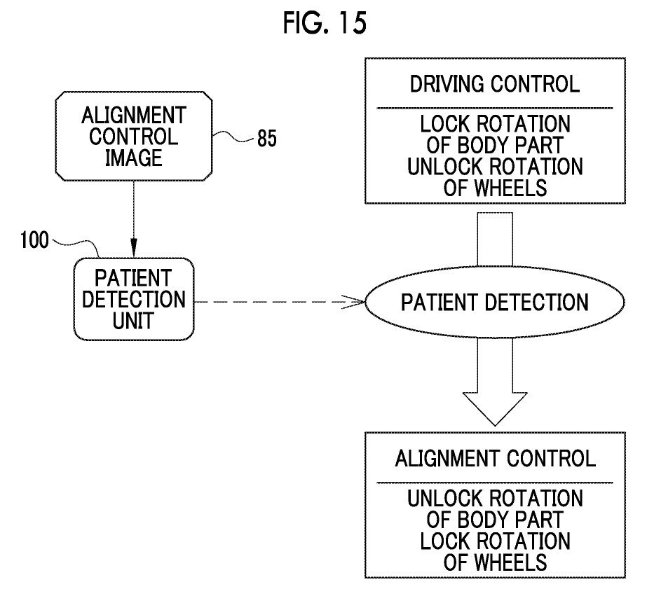

In the first embodiment, in a case where the self-position PS is the target position TP, the rotation of the body part 14 by the first locking mechanism 36 is released, and the rotation of the wheels 15 is locked by the second locking mechanism 54, but the present disclosure is not limited thereto. As an example, the second embodiment may be as shown in FIGS. 14 and 15.

As shown in FIG. 14, the processor 45 of the present embodiment functions as a cassette detection unit 95 in addition to each processing unit of the first embodiment. The alignment control image 85 is input to the cassette detection unit 95. The cassette detection unit 95 detects whether or not the electronic cassette 12 is shown in the alignment control image 85 by using an image recognition technology.

In a case where the electronic cassette 12 is detected to be shown in the alignment control image 85 by the cassette detection unit 95, the processor 45 of the present embodiment releases the locking of the rotation of the body part 14 by the first locking mechanism 36, locks the rotation of the wheels 15 by the second locking mechanism 54, and transitions from the driving control to the alignment control. In a case where the electronic cassette 12 is detected to be shown in the alignment control image 85, it is an example of a "case where a set condition is satisfied" according to the disclosed technology. The cassette detection unit 95 may detect whether or not the electronic cassette 12 is shown in the alignment control image 85 by using the trained model such as the cassette contour extraction model 86 of the first embodiment.

Alternatively, as shown in FIG. 15, the processor 45 of the present embodiment functions as a patient detection unit 100 in addition to each processing unit of the first embodiment. The alignment control image 85 is input to the patient detection unit 100. The patient detection unit 100 detects whether or not the patient P is shown in the alignment control image 85 by using an image recognition technology. In a case where the patient P is detected to be shown in the alignment control image 85 by the patient detection unit 100, the processor 45 of the present embodiment releases the locking of the rotation of the body part 14 by the first locking mechanism 36, locks the rotation of the wheels 15 by the second locking mechanism 54, and transitions from the driving control to the alignment control. In a case where the patient P is detected to be shown in the alignment control image 85, it is an example of a "case where a set condition is satisfied" according to the disclosed technology. In a case where the electronic cassette 12 or the patient P is detected to be shown in the alignment control image 85 in this way, the rotation of the body part 14 by the first locking mechanism 36 is released, and the rotation of the wheels 15 is locked by the second locking mechanism 54, so that the body part 14 can be rotated at an appropriate timing and the rotation of the wheels 15 can be locked, and the alignment control can be executed without a hitch.

### Third Embodiment

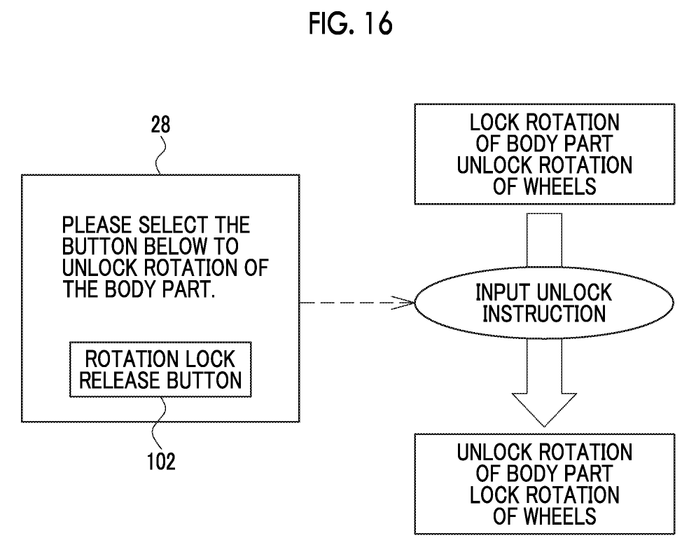

As shown in FIG. 16 as an example, in the present embodiment, in a case where the self-position PS is the target position TP, the processor 45 displays a rotation lock release button 102 on the operation panel 28. In a case where the rotation lock release button 102 is selected by the operator OP and the unlock instruction is input, the processor 45 releases the locking of the rotation of the body part 14 by the first locking mechanism 36 and locks the rotation of the wheels 15 by the second locking mechanism 54. In a case where the unlock instruction is input, it is an example of a "case where a set condition is satisfied" according to the disclosed technology. The unlock instruction is an example of an "operation instruction for unlocking" according to the disclosed technology. With such a configuration, the body part 14 can be rotated at an appropriate timing and the rotation of the wheels 15 can be locked, and the alignment control can be executed without a hitch. The rotation lock release button 102 may be provided as a physical button on the radiation source 13, the body part 14, or the like.

### Fourth Embodiment

In the first embodiment, the contour OLC of the electronic cassette 12 is extracted from the alignment control image 85, but the present disclosure is not limited thereto. As an example, the fourth embodiment may be as shown in FIG. 17.

In FIG. 17, the processor 45 of the present embodiment functions as a joint point extraction unit 105 instead of the cassette contour extraction unit 80 of the first embodiment. The alignment control image 85 is sequentially input to the joint point extraction unit 105. The joint point extraction unit 105 extracts the joint point J of the patient P from the alignment control image 85 by using a joint point extraction model 106. FIG. 17 illustrates a case where both shoulder joints, both elbow joints, and both hip joints are extracted as the joint points J. According to the line connecting each joint point J, the center IC of the imaging part of the patient P and the posture of the patient P with respect to the radiation source 13 are known. The joint point extraction model 106 is a trained model configured by, for example, a convolutional neural network, similarly to the cassette contour extraction model 86. The joint point extraction unit 105 outputs a joint point extraction result 107 to the alignment control unit 82.

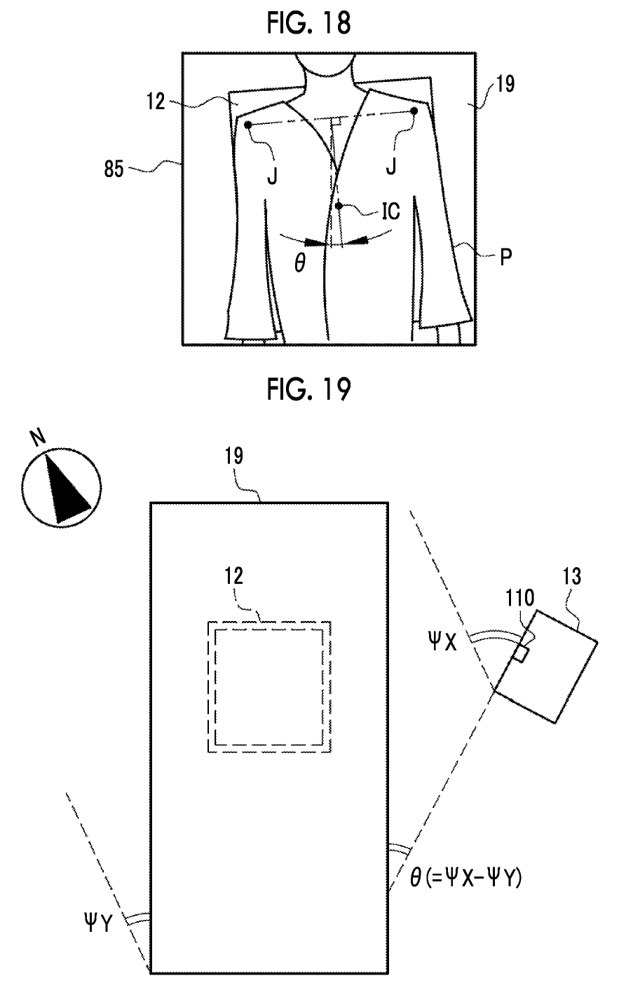

In a case where the posture of the radiation source 13 is inclined with respect to the patient P, the alignment control unit 82 drives the rotation mechanism 35 to rotate the body part 14, thereby rotating the radiation source 13 to eliminate the inclination. As shown in FIG. 18 as an example, the inclination angle θ in this case is an angle between a side extending to the left and right of the radiation source 13 and a perpendicular line passing through the center IC of the imaging part of the patient P and bisecting a line connecting the two shoulder joints, that is, the body axis (head-tail axis) of the patient P. In addition, in a case where the irradiation center RC of the radiation R and the center IC of the imaging part of the patient P are shifted, the alignment control unit 82 controls the driving of the alignment actuator 52 to move the radiation source 13 to eliminate the shift. By extracting the joint point J of the patient P from the alignment control image 85 in this way, it is possible to contribute to the higher-accuracy alignment between the radiation source 13 and the electronic cassette 12. In this case, in a case where the long side of the decubitus imaging table 19 and the head-tail axis of the patient P are not parallel to each other and the irradiation center RC of the radiation R and the center IC of the imaging part of the patient P are shifted, the shift may be eliminated by moving the radiation generation apparatus 11 along the head-tail axis of the patient P (moving the radiation generation apparatus 11 obliquely with respect to the long side of the decubitus imaging table 19).

### Fifth Embodiment

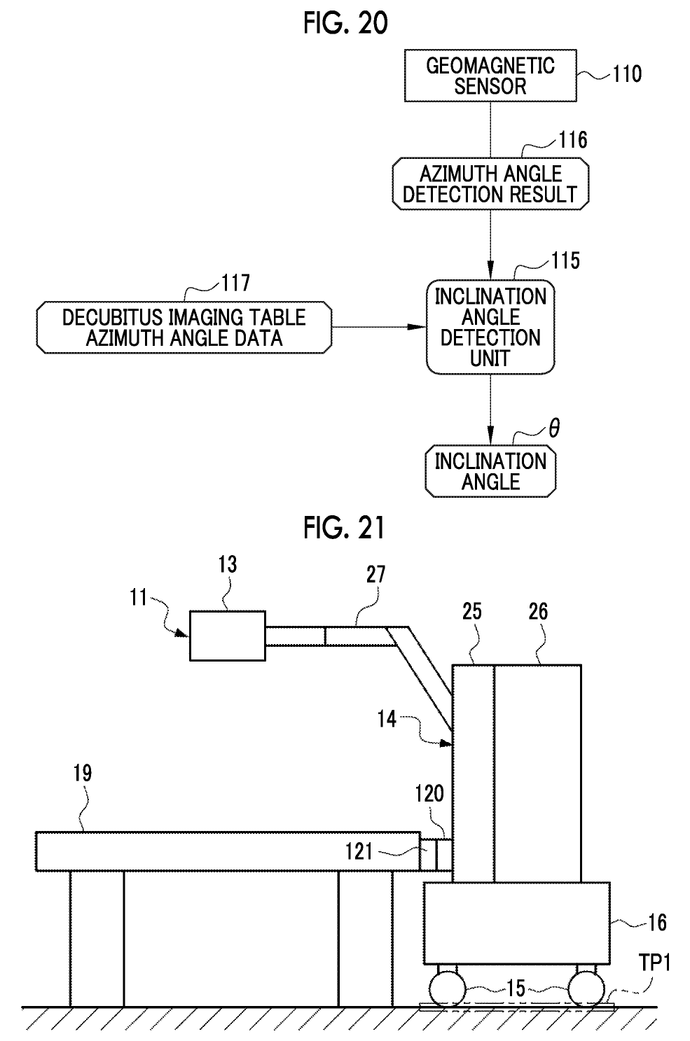

As shown in FIGS. 19 and 20 as an example, the inclination angle θ may be detected based on the output from a geomagnetic sensor 110 provided in the radiation source 13.

In FIG. 20, the processor 45 of the present embodiment functions as an inclination angle detection unit 115 in addition to each processing unit of the first embodiment. An azimuth angle detection result 116 of the radiation source 13 from the geomagnetic sensor 110 and decubitus imaging table azimuth angle data 117 are input to the inclination angle detection unit 115. The azimuth angle detection result 116 includes an angle ΨX shown in FIG. 19. The angle ΨX is an angle between a side extending to the left and right of the radiation source 13 and magnetic north around the vertical axis VA. The decubitus imaging table azimuth angle data 117 includes an angle ΨY shown in FIG. 19. The angle ΨY is an angle between the long side of the decubitus imaging table 19 and magnetic north around the vertical axis VA. The decubitus imaging table azimuth angle data 117 is registered in advance in the storage 46 as the data for alignment control of the control data 48 by the administrator or the like of the hospital. The angle ΨX of the azimuth angle detection result 116 is an example of a "azimuth angle of the body part" according to the disclosed technology. The angle ΨY of the decubitus imaging table azimuth angle data 117 is an example of a "azimuth angle of the imaging table" according to the disclosed technology.

The inclination angle detection unit 115 calculates a difference (ΨX - ΨY) between the angle ΨX of the azimuth angle detection result 116 and the angle ΨY of the decubitus imaging table azimuth angle data 117. The inclination angle detection unit 115 outputs the calculated difference to the alignment control unit 82 as the inclination angle θ.

As described above, in the fifth embodiment, the inclination angle detection unit 115 derives the difference between the azimuth angle ΨX of the radiation source 13 detected by the geomagnetic sensor 110 and the azimuth angle ΨY of the decubitus imaging table 19 as the inclination angle θ. Therefore, the inclination angle θ can be easily detected as compared with the first embodiment and the fourth embodiment in which the inclination angle θ is detected based on the alignment control image 85.

The inclination angle θ may be derived based on the output of the sensor such as the rotary encoder that detects the rotation direction and the rotation amount of the wheels 15. In this case, the inclination angle θ is derived by obtaining, from the output of the sensor such as the rotary encoder, the orientation of the radiation generation apparatus 11 at the current time by driving on which path from the standby position HP with reference to the angle with the long side of the decubitus imaging table 19 at the standby position HP. Alternatively, a marker may be attached to the long side of the decubitus imaging table 19, the marker may be imaged by the camera provided in the body part 14, and the inclination angle θ may be derived from the distortion of the pattern of the marker shown in the captured image. Furthermore, a distance sensor such as a time-of-flight (TOF) sensor may be provided in the body part 14, and the inclination angle θ may be derived based on a difference (DPb - DPb) between distances DP1 and DP2 from the distance image output by the distance sensor to two points P1 and P2 set in advance on the long side of the decubitus imaging table 19.

### Sixth Embodiment

As shown in FIG. 21 as an example, in the present embodiment, a first magnet 120 is attached to the body part 14, and a second magnet 121 is attached to the decubitus imaging table 19. The first magnet 120 is disposed at a front surface center of the front part 25 of the body part 14 at a height that matches the long side of the decubitus imaging table 19. The second magnet 121 is disposed at a position facing the first magnet 120 in a case where the radiation generation apparatus 11 is at the first target position TP1 and the inclination angle θ is 0° on the long side of the decubitus imaging table 19. The first magnet 120 is an electromagnet, and a magnetic field is generated by energization and disappears by de-energization. The processor 45 cuts off the energization of the first magnet 120 until the radiation generation apparatus 11 reaches the first target position TP1, and starts the energization of the first magnet 120 in a case where the radiation generation apparatus 11 reaches the first target position TP1.

In a case where the radiation generation apparatus 11 reaches the first target position TP1 and the rotation of the body part 14 by the first locking mechanism 36 is released, the first magnet 120 is attracted to the second magnet 121, so that the alignment between the radiation source 13 and the decubitus imaging table 19 is performed. As a result, it is possible to save the effort of processing of detecting the inclination angle θ or driving the rotation mechanism 35 to eliminate the inclination.

### Seventh Embodiment

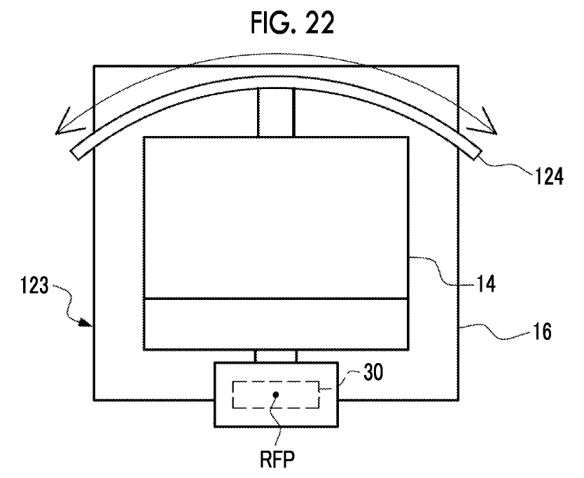

In the first embodiment, the body part 14 is rotated around the vertical axis VA passing through the center MC of the body part 14, but the present disclosure is not limited thereto. As shown in a radiation generation apparatus 123 shown in FIG. 22 as an example, the body part 14 may be rotated around the vertical axis VA passing through a focal point RFP of the radiation R. In this case, the rotation mechanism rotates the body part 14 along an arc-shaped guide rail 124 erected on the carriage unit 16 with the focal point RFP as the center. In this case, since the position of the focal point RFP does not change even in a case where the body part 14 is rotated, the alignment between the radiation source 13 and the electronic cassette 12 can be easily performed. The body part 14 may be rotated around the vertical axis VA passing through both the center MC of the body part 14 and the focal point RFP of the radiation R.

### Eighth Embodiment

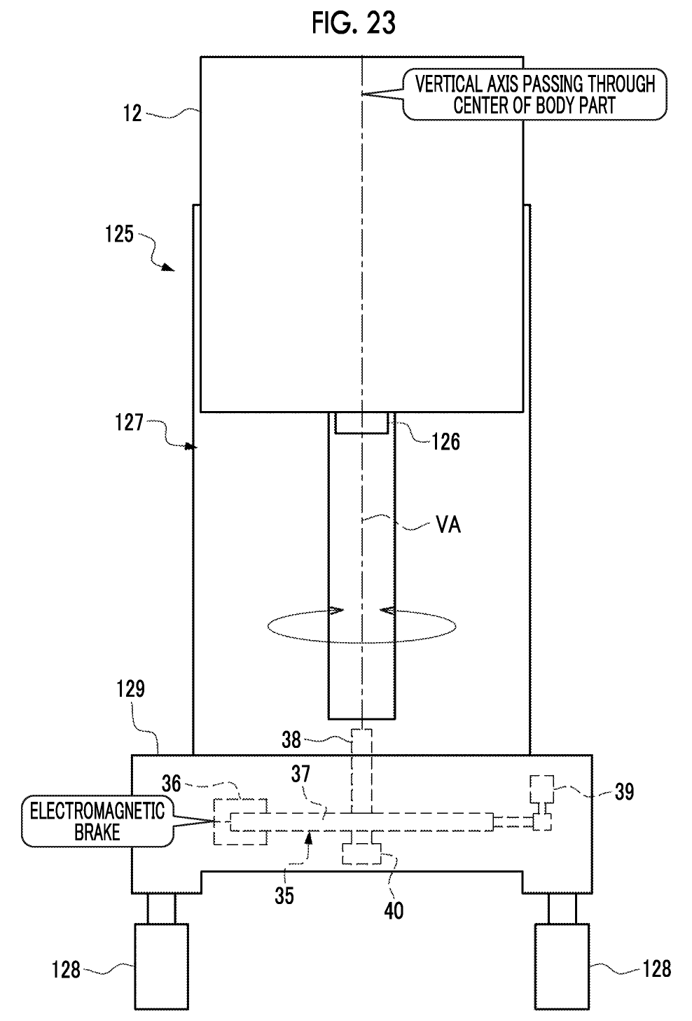

In each of the above-described embodiments, the radiation generation apparatus 11 comprising the rotation mechanism 35 has been described, but the present disclosure is not limited thereto. As shown in FIG. 23 as an example, the rotation mechanism 35 may be applied to a driving imaging table 125. The imaging table 125 has a configuration in which a body part 127 having an arm 126 that holds the electronic cassette 12 at a distal end is mounted on a carriage unit 129 having wheels 128, and the rotation mechanism 35 is built in the carriage unit 129.

The electronic cassette 12 also has a relatively large weight of several kilograms, like the radiation source 13. Therefore, with such a configuration, the body part 127 does not move due to a reaction force, and the arm 126 does not stop at an unintended position due to inertia, as in a case where the arm 126 is rotated, so that the rotation of the electronic cassette 12 can be stabilized. Therefore, it is possible to contribute to the high-accuracy alignment between the patient P and the electronic cassette 12 and between the radiation source 13 and the electronic cassette 12.

In each of the above-described embodiments, the decubitus imaging table 19 has been described as an example, but the present disclosure is not limited thereto. The body part 14 may be rotated to eliminate the inclination with respect to the upright imaging table 20.

An example in which the radiation generation apparatus 11 autonomously drives has been described, but the present disclosure is not limited thereto. The operator OP may manually drive the radiation generation apparatus 11. In addition, an example in which the body part 14 is automatically rotated by controlling the driving of the rotation mechanism 35 has been described, but the present disclosure is not limited thereto. The operator OP may manually rotate the body part 14.

A guide rail may be provided on the long side of the decubitus imaging table 19, a protrusion connected to the guide rail may be provided in the body part 14, and the radiation generation apparatus 11 may be moved along the guide rail.

The top plate of the decubitus imaging table 19 may be configured to be rotatable, not only the body part 14. The top plate may be rotated to eliminate the inclination.

The alignment control image 85 may be displayed on the operation panel 28. In this case, the contour OLC of the electronic cassette 12, the center CC of the detection surface 17 of the electronic cassette 12, the irradiation center RC of the radiation R, and the like may be superimposed and displayed on the alignment control image 85 as marks.

The camera 32 may be attached to the arm 27 instead of the radiation source 13. Similarly, the camera 33 may be provided in the body part 14 instead of the carriage unit 16. A plurality of the cameras 32 and 33 may be provided.

The camera 32 and the camera 33 may be integrated into one camera. In this case, the orientation of the camera is set to an orientation in which the driving control image 70 can be captured in a case where the driving control is executed, and is set to an orientation in which the alignment control image 85 can be captured in a case where the alignment control is executed.

The environment information sensor is not limited to the example of the camera 33. A light detection and ranging (LiDAR) sensor, a TOF sensor, or the like may be used. In addition, an inertial measurement unit (IMU) in which an acceleration sensor and a gyro sensor are combined, an ultrasonic sensor, a radar sensor, a magnetic sensor, or the like may be used.

The radiographic image detection device is not limited to the example of the electronic cassette 12. A computed radiography (CR) cassette may be used. In addition, the subject is not limited to the example of the patient P. The subject may be a diseased animal such as a dog or a cat.

In each of the above-described embodiments, for example, each processing of each processing unit such as the image acquisition unit 60, the feature point extraction unit 61, the self-position estimation/map data creation unit 62, the driving control unit 63, the cassette contour extraction unit 80, the radiation source position/posture derivation unit 81, the alignment control unit 82, the cassette detection unit 95, the patient detection unit 100, the joint point extraction unit 105, and the inclination angle detection unit 115 is executed by any computer. In addition, any computer may execute these types of processing by a processor as hardware, a program as software, or a combination thereof. In such a case, the processor is configured to execute various types of processing in each of the above-described embodiments in cooperation with the program, and may function as each unit or each means in each of the above-described embodiments. In addition, the execution order of the processing by the processor is not limited to the above-described order and may be changed as appropriate. Any computer may be a general-purpose computer, a computer for specific use, a workstation, or another system capable of executing each processing.

The processor may be configured using one or more pieces of hardware, and the type of hardware is not limited. For example, the processor may be configured by an example of the CPU or a micro processing unit (MPU), a programmable logic device such as a field programmable gate array (FPGA), a dedicated circuit for executing specific processing such as an application specific integrated circuit (ASIC), a graphics processing unit (GPU), a neural processing unit (NPU), or the like. The types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is configured by an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium or other storage). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The technologies according to the following supplementary notes can be understood from the above description.

### Supplementary Note 1

A radiation generation apparatus comprising:

a body part having an arm that holds, at a distal end, a radiation source that emits radiation toward a subject, the body part being mounted on a carriage unit having wheels; and

a rotation mechanism that allows the body part to rotate around a vertical axis with respect to the carriage unit.

### Supplementary Note 2

The radiation generation apparatus according to Supplementary Note 1, further comprising:

a first locking mechanism that is capable of locking the rotation of the body part by the rotation mechanism.

### Supplementary Note 3

The radiation generation apparatus according to Supplementary Note 2,

wherein the locking by the first locking mechanism is released in a case where a set condition is satisfied.

### Supplementary Note 4

The radiation generation apparatus according to Supplementary Note 3,

wherein the locking by the first locking mechanism is released in a case where a self-position is a target position.

### Supplementary Note 5

The radiation generation apparatus according to Supplementary Note 4, further comprising:

an environment information sensor; and

a processor,

wherein the processor creates map data of a surrounding environment while estimating the self-position from output data of the environment information sensor.

### Supplementary Note 6

The radiation generation apparatus according to Supplementary Note 5,

wherein the radiation generation apparatus is capable of autonomous driving based on the map data.

### Supplementary Note 7

The radiation generation apparatus according to Supplementary Note 3, further comprising:

a camera,

wherein the locking by the first locking mechanism is released in a case where it is detected that a radiographic image detection device or the subject is shown in an image of the camera.

### Supplementary Note 8

The radiation generation apparatus according to Supplementary Note 3,

wherein the locking by the first locking mechanism is released in a case where an operation instruction for releasing the locking is input.

### Supplementary Note 9

The radiation generation apparatus according to any one of Supplementary Notes 2 to 8,

wherein the carriage unit has a second locking mechanism that is capable of locking rotation of the wheels.

### Supplementary Note 10

The radiation generation apparatus according to Supplementary Note 9,

wherein the locking by the second locking mechanism is performed in a case where the locking by the first locking mechanism is released.

### Supplementary Note 11

The radiation generation apparatus according to any one of Supplementary Notes 2 to 10,

wherein the first locking mechanism is an electromagnetic brake.

### Supplementary Note 12

The radiation generation apparatus according to any one of Supplementary Notes 1 to 11, further comprising:

a processor,

wherein the processor controls the rotation of the body part by the rotation mechanism based on an inclination angle between the radiation source and a radiographic image detection device, an imaging table, or the subject.

### Supplementary Note 13

The radiation generation apparatus according to Supplementary Note 12,

wherein the processor derives a difference between an azimuth angle of the radiation source detected by a geomagnetic sensor and an azimuth angle of the imaging table as the inclination angle.

### Supplementary Note 14

The radiation generation apparatus according to any one of Supplementary Notes 1 to 13,

wherein a first magnet is provided in the body part, and

alignment between the radiation source and the imaging table is performed by attraction of the first magnet to a second magnet provided on the imaging table.

### Supplementary Note 15

The radiation generation apparatus according to any one of Supplementary Notes 1 to 14,

wherein the rotation mechanism rotates around the vertical axis passing through at least any of a center of the body part or a focal point of the radiation.

In addition, the technologies described in the following supplementary note can be understood from the description of the eighth embodiment.

### Supplementary Note 16

An imaging table comprising:

a body part having an arm that holds a radiographic image detection device at a distal end, the body part being mounted on a carriage unit having wheels; and

a rotation mechanism that allows the body part to rotate around a vertical axis with respect to the carriage unit.

The technology of the present disclosure can also be combined with various embodiments and/or various modification examples described above, as appropriate. In addition, it goes without saying that the present disclosure is not limited to each of the embodiments described above, various configurations can be adopted as long as the configuration does not deviate from the gist. Furthermore, the technology of the present disclosure extends to a storage medium that non-transitorily stores the program, and a computer program product including the program, in addition to the program.

The above-described contents and the above-shown contents are the detailed description of the parts according to the technology of the present disclosure, and are merely an example of the technology of the present disclosure. For example, the above description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate grasping the parts according to the technology of the present disclosure, in the above-described contents and the above-shown contents, the description of technical general knowledge and the like that do not particularly require description for enabling the implementation of the technology of the present disclosure are omitted.

In the present specification, "A and/or B" has the same meaning as "at least one of A or B". That is, "A and/or B" means that it may be only A, only B, or a combination of A and B. In addition, in the present specification, also in a case where three or more matters are expressed in association by "and/or", the same concept as "A and/or B" is applied.

All of the documents, the patent applications, and the technical standards described in the present specification are incorporated herein by reference to the same extent as in a case where each of the documents, patent applications, and technical standards is specifically and individually described by being incorporated by reference.

What is claimed is:

1. A radiation generation apparatus comprising:
a body part having an arm that holds, at a distal end, a radiation source that emits radiation toward a subject, the body part being mounted on a carriage unit having wheels; and
a rotation mechanism that allows the body part to rotate around a vertical axis with respect to the carriage unit.

2. The radiation generation apparatus according to claim 1, further comprising:
a first locking mechanism that is capable of locking the rotation of the body part by the rotation mechanism.

3. The radiation generation apparatus according to claim 2,
wherein the locking by the first locking mechanism is released in a case where a set condition is satisfied.

4. The radiation generation apparatus according to claim 3,
wherein the locking by the first locking mechanism is released in a case where a self-position is a target position.

5. The radiation generation apparatus according to claim 4, further comprising:
an environment information sensor; and
a processor,
wherein the processor creates map data of a surrounding environment while estimating the self-position from output data of the environment information sensor.

6. The radiation generation apparatus according to claim 5,
wherein the radiation generation apparatus is capable of autonomous driving based on the map data.

7. The radiation generation apparatus according to claim 3, further comprising:
a camera,
wherein the locking by the first locking mechanism is released in a case where it is detected that a radiographic image detection device or the subject is shown in an image of the camera.

8. The radiation generation apparatus according to claim 3,
wherein the locking by the first locking mechanism is released in a case where an operation instruction for releasing the locking is input.

9. The radiation generation apparatus according to claim 2,
wherein the carriage unit has a second locking mechanism that is capable of locking rotation of the wheels.

10. The radiation generation apparatus according to claim 9,
wherein the locking by the second locking mechanism is performed in a case where the locking by the first locking mechanism is released.

11. The radiation generation apparatus according to claim 2,
wherein the first locking mechanism is an electromagnetic brake.

12. The radiation generation apparatus according to claim 1, further comprising:
a processor,
wherein the processor controls the rotation of the body part by the rotation mechanism based on an inclination angle between the radiation source and a radiographic image detection device, an imaging table, or the subject.

13. The radiation generation apparatus according to claim 12,
wherein the processor derives a difference between an azimuth angle of the radiation source detected by a geomagnetic sensor and an azimuth angle of the imaging table as the inclination angle.

14. The radiation generation apparatus according to claim 1,
wherein a first magnet is provided in the body part, and
alignment between the radiation source and the imaging table is performed by attraction of the first magnet to a second magnet provided on the imaging table.

15. The radiation generation apparatus according to claim 1,
wherein the rotation mechanism rotates around the vertical axis passing through at least any of a center of the body part or a focal point of the radiation.
